(19) United States
(12) Patent Application Publication
(10) Pub. No.: US 2024/0407661 A1
(43) Pub. Date: Dec. 12, 2024

(54) BIOLOGICS DETECTION DEVICE

(71) Applicants: Nassar O. Said, Naur (JO); Adeeb Al-Zoubi, Lake Worth, FL (US); Advanced Medical Solutions International, LLC, Lake Worth, FL (US)

(72) Inventors: Nassar O. Said, Nassar (JO); Adeeb : Al-Zoubi, Lake Worth, FL (US)

(21) Appl. No.: 18/273,970

(22) PCT Filed: Jan. 25, 2022
(86) PCT No.: PCT/US2022/013690

§ 371 Date: Jul. 24, 2023

Related U.S. Application Data
(60) Provisional application No. 63/141,313, filed on Jan. 25, 2021.

(51) Int. Cl. A61B 5/0507; A61B 5/00; G01N 21/17; G01N 21/21; G01N 22/00

(52) U.S. Cl. CPC A61B 5/0507; A61B 5/0082; G01N 21/21; G01N 22/00; G01N 2021/1789; G01N 2333/165

(57) ABSTRACT

Methods and apparatus for non-invasively and accurately detecting a chemically-stable molecular structure. The methods and apparatus can be used to detect molecules associated with disease and disorders, and objects or subjects associated with such disease and disorders, such as contaminated with the molecules or pathogens, or having disease or disorder associated with the molecules or pathogens.

### RELATED APPLICATIONS

This application claims priority under 35 U.S.C. § 119(e) to U.S. Provisional Application, U.S. Ser. No. 63/141,313, filed on Jan. 25, 2021, the content of which is incorporated herein by reference in its entirety.

### BACKGROUND

The dielectric properties of organic molecules have received much interest some decades ago for many reasons. For instance, they determine the pathways of current flows through those molecules and, thus, are very important in the analysis of a wide range of biomedical applications, knowledge of these electrical properties can lead to an understanding of the underlying basic biological processes, and biological relaxation period of studies of organic molecules have long been important in electrophysiology and biophysics.

### SUMMARY

Methods and apparatus for non-invasively and accurately detecting a chemically-stable molecular structure. For example, some embodiments are configured to perform a non-invasive remote technique that is capable of accurate and quick diagnosis for COVID-19 (e.g., severe acute respiratory syndrome coronavirus 2 (SARS-COV-2)) infected persons using a combination of radiofrequency (RF) and infrared (IR) electromagnetic waves. Signals reflected from a tested medium are analyzed to detect a characteristic relaxation time period for the chemically-stable molecule.

Application of an external electromagnetic field to a biological organic molecular structure induces an absolute and relative redistribution of internal charges with respect to the field lines. This process is characterized by the “relaxation time” time of the system. There are different types of relaxation, which define material properties and are expressed upon application of alternating electric fields. The most common are dipole relaxation and ionic, atomic and electron polarization, and they are ordered according to their resonant frequencies. The dominance of any one of these types of relaxation in a whole system depends on the frequency of the external stimulus. In practice, many other types of relaxation are observed, such as boundary effects and complex molecular vibrations. In the simplest model, external fields cause alignment of the molecules along their tension lines, and as the field direction alternates, molecules follow these changes. At some point, the frequency of the alternating field is so rapid that molecules or any other field-sensitive entities cannot follow the directionality change.

When the external field frequency matches the relaxation time of the molecular structure under investigation, such system behavior is called the simple Debye model, and it is a simplistic representation of the actual process. Representation of the dielectric permittivity is a complex form of the real and imaginary part which allows one to describe the fundamental properties of tissue to accumulate and/or dissipate the energy of electromagnetic irradiation.

To analyze the response of a particular organic molecule to electromagnetic filed stimulation, one needs data on the specific conductivity and relative permittivity of the molecule and information about the how atoms are connected in the organic molecular structure. A microscopic description of the response is complicated by the variety of atoms shapes and their distribution inside the molecular structure.

Therefore, a macroscopic approach is always a net result of several microscopic phenomena and is most often used to characterize field distributions not only in organic molecular structure but also in biological systems as general.

The description that follows shows that the macroscopic electric and dielectric measurements of organic molecular structure properties are due to several microscopic factors: molecule structure inhomogeneity (intrinsic internal structure), physiological state of the organic molecule, and other factors that could play a role for the so called crossover frequency (CoF) which has a significant role on the relaxation time of the molecular structure under investigation.

Electrical properties; quantitatively reflects the contents of the molecule building block, that is the different chemical compounds and biomaterials in vivo or in vitro those affect drastically the electrical properties of the organic molecule structure. For example, plasma contains thousands of different substances, such as proteins, glucose, salts, vitamins, hormones, and antibodies.

### BRIEF DESCRIPTION OF THE DRAWINGS

FIG. 1 shows a basic block diagram of an embodiment

FIG. 2 shows the device in operation: (a) manual scanning; (b) vertical scanning.

FIG. 3 shows dispersion regions of an ideal organic molecular structure.

FIG. 4 shows the real parts of the permittivity for a biological molecular structure.

FIG. 5 shows (a) the dielectric constant of the measured data and data the fitted models; (b) the conductivity of the measured and the fitted models.

FIG. 6 shows the mechanism of two resonators oscillatory system under the effect of frequency pulling.

FIG. 7 shows general response curves and microstrip fabrication for transmitting and receiving resonance antennas.

FIG. 8 shows coding of IR beam and RF transmitted signal to provide transmitted RF pulses with desired relaxation period.

Below is provided a brief discussion about electrical properties of biological molecular structure and the effect of the applied field on them.

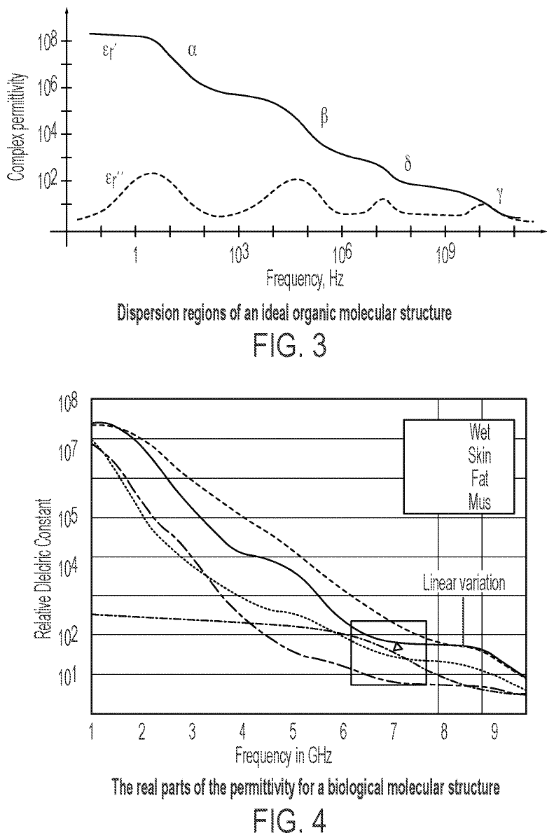

FIG. 3 shows the changes in complex permittivity of biological molecular structure as frequency changes (called dispersion). These regions have been named as α, β, γ, and δ. The α, δ dispersions region correspond for the lowest and highest operating frequency. But the β-dispersion region falls in the microwave range as well as the δ-dispersion region.

In general, complex permittivity (function of frequency) of molecular structure is:

ε=ε₀\hat{ε}ᵣ(ω); Where \hat{ε}ᵣ(ω)=\acute{ε}ᵣ(ω)−j\tilde{ε}ᵣ(ω)) (1)
εᵣ is called the dielectric constant or the relative permittivity; and quantifies the dissipation of energy within the material.

A example model to estimate properties of dielectrics is Debye model as given by equation (2).

ε(ω)=ε_{∞}+ΣₙΔεₙ/1+jωτₙ+σ/jωε₀ (2)
where, τ is the time constant ε_{∞} is the permittivity when ωτ>>1 or at high frequencies, σ is the conductivity of the organic molecular structure and ω is the angular frequency of the applied field.

The Debye model was slightly modified by introducing a broadening factor introducing the Cole-Cole model. The Cole-Cole model is as in equation (3).

ε(ω)=ε_{∞}+ΣₙΔεₙ/(1+jωτₙ)^{1-α}^{ₙ}+σ/jωε₀ (3)
α is called the distribution parameter.

The real parts of the permittivity for biological molecular structure are plotted in FIG. 4.

The Cole-Cole Model of equation (3) offers an efficient and accurate representation of many types of biological molecular structure over a very wide frequency band and has been recently used to reduce the complexity of the experimental data obtained for various human tissues (brain, fat, breast, skin, bone, liver etc.).

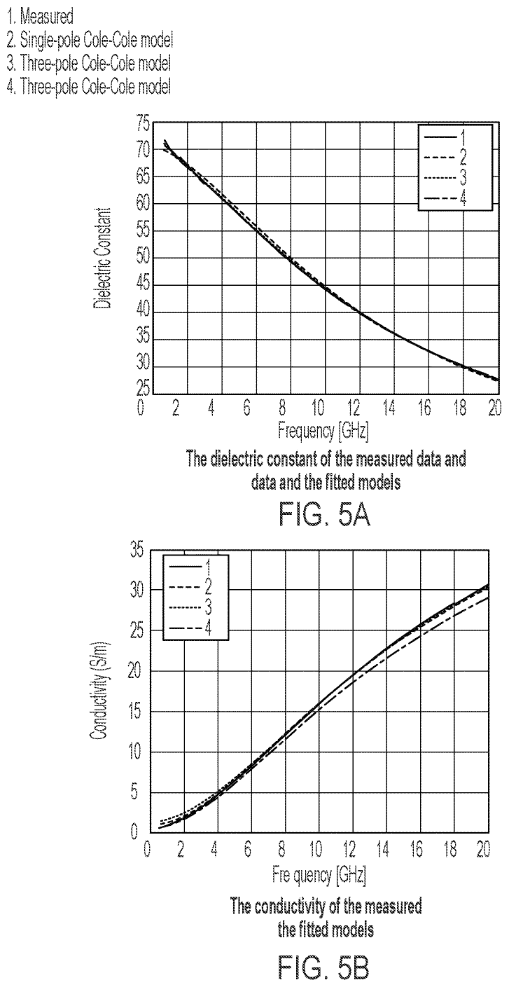

The parameters α, and n are fixed at 0.1 in all three models. The dielectric constant and the conductivity of the sample and the fitted models are shown in FIG. 5(a) and FIG. 5(b) respectively. As shown, the single-pole model may be sufficient to represent this data with less number of parameters and computational time.

Generally, the application of a sudden EM field to a molecular structure causes the charges spins, internal current distribution, and dipoles within the structure to respond to the applied fields to form an average field based on the naturally existed local field. The total field includes the effects of both the applied field, transients, and the charged-particles fields that cause depolarization fields. This will cause the system to be in non-equilibrium for a period of time (Relaxation). For example, when an applied EM field interacts with a dielectric material, the dipoles reorient and charge moves, so that the local fields in the material are modified by surface charge dipole depolarization fields that oppose the applied field.

If the applied EM field is static, then an equilibrium state will be reached after some time. However, if the applied field is a time-variant function, then the molecular structure will continuously relax as long as the time-variant field is applied, but with a time lag. Dielectric relaxation can be a result of dipolar and induced polarization, lattice-phonon interactions, defect diffusion, or the motion of free charges. Time-dependent EM fields produce non-equilibrium behavior in the materials. However, for homogenous molecular structure and time-harmonic EM fields, when the response is averaged over a cycle, the dynamic re-adjustment of the molecules in response to the EM field is called relaxation and is distinct from resonance.

Dielectric relaxation times are related to how the dipole moments and charge are constrained by the surrounding molecular structures. The characteristic relaxation time for a polarized material that was in an applied EM field at t=0 to decay to a steady state is related to the coupling between dipoles and details of the lattice structure. At high frequencies, the electric response of a material lags behind the applied EM field as the EM field changes faster than the relaxation response of the molecules. This lag is due to long and short-range forces and inertia. Basically, the relaxation time is a function of many molecular parameters among them is the Dielectric Constant. Êᵣ(ω)=\acute{ε}ᵣ(ω)−j\tilde{ε}ᵣ(ω), and the complex conductivity σ* of the molecular structure under investigation.

### DETAILED DESCRIPTION OF CERTAIN EMBODIMENTS

Some embodiments of the invention are described in greater detail with reference to the accompanying drawings. In the following description, the same drawing reference numerals are used for the same elements even in different drawings.

Some embodiments relate to stimulation of a biological molecular structure using time-varying electromagnetic energy and detecting a signal in response to the stimulation based on the electrical parameters (dielectric constant, conductivity, and relaxation time) of the biological molecular structure.

Some embodiments rely on the relaxation time determined from a number of surely infected specimens contaminated with the COVID-19 virus biological molecular structure. The relaxation time may be stored within the memory area of a device configured in accordance with the techniques described herein.

Some embodiments rely on the property of frequency pulling of multiple controlled oscillators where a change in the frequency of an oscillator occurs due to a change in detected relaxation time of the biological molecular structure under investigation.

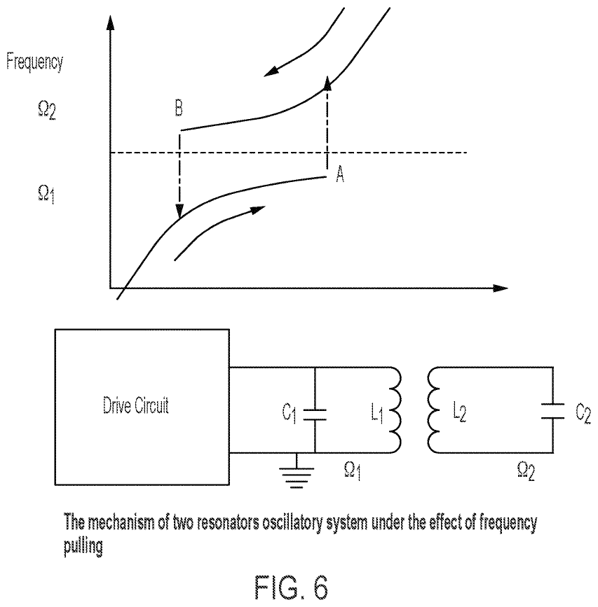

Frequency pulling takes place when the frequencies (Ω1) and (Ω2) (FIG. 6) are close together. Frequency pulling is used to stabilize the frequency of single-resonator oscillators. In some embodiments, the oscillator is connected to a supplementary resonator with a high quality (Q) factor. Owing to the frequency, pulling the oscillator frequency ω is maintained close to the frequency of the supplementary resonator Ω2.

The frequency of self-oscillations in a two-resonator oscillatory system (FIG. 6) depends on the direction of the change in the tuning of one of the resonators. As Ω1 increases, the oscillator frequency ω increases from O toward A, and when Ω1 decreases, ω decreases from C toward B. At the points A and B an abrupt change in the frequency occurs (the dotted arrows).

Some embodiments use this phenomenon to load the tuning cavities of the transmitter and receiver and to pull the oscillation frequency from one to another depending on the stimulated molecular structure of COVID-19 virus calculated relaxation time compared with stored reference data.

As discussed above, relaxation time is a function that relies greatly on the frequency dependent parameter ε(ω) of the molecular structure of a biological molecular structure (e.g., the COVID-19 virus).

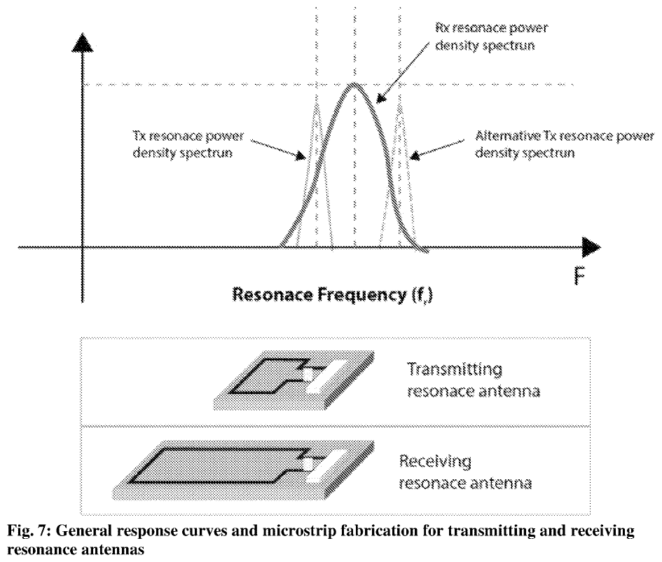

Some embodiments include two cylindrical resonance-cavity backed with microstrip resonator antennas constructed using precise shape and orientation of microstrip fabrication to give the general response curves and as shown in FIG. 7.

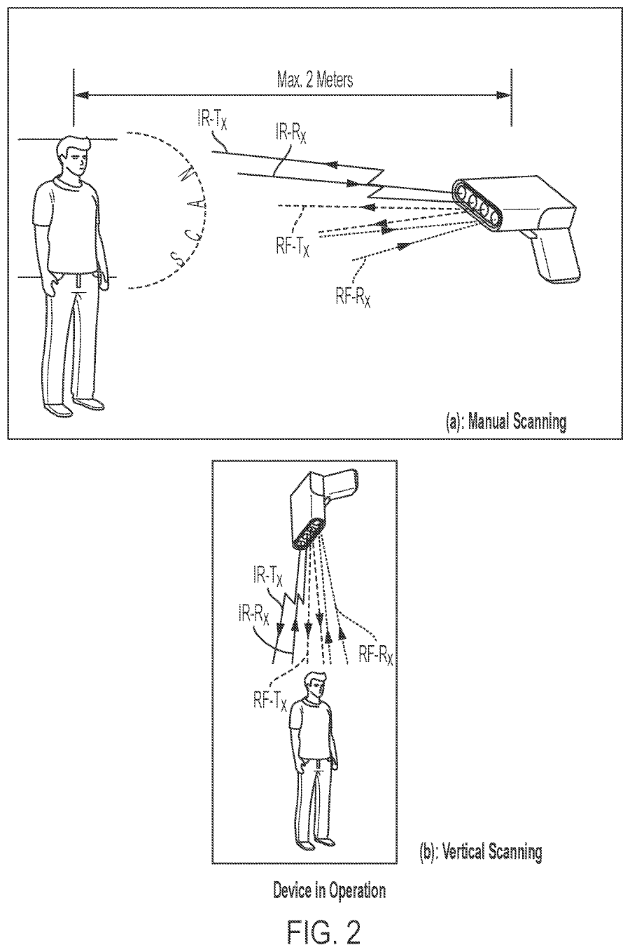

The two microstrip resonance cavities (transmitting and receiving antennas) shown in FIG. 7 are designed with a high quality factor (Q) for the receiving antenna and very high (Q) for the transmitting antenna The physical layout of that antenna is implemented to keep zero coupling coefficient (k) between them. In some embodiments, both antennas have directivity pattern perpendicular to their plane as they are placed at base of cylindrical cavities. FIG. 2 illustrates the test methodology.

From basic principles and for each resonator cavity:

$$Q = \frac{{ω}_{0}}{R} {L}_{i} = \frac{1}{R} \sqrt{\frac{{L}_{i}}{C}} \qquad ( 4 )$$

where Ω₀: Self resonant frequency; i=1, or 2; L₁: self-inductance of transmitter L₂: self-inductance of receiver; C: shunt tuning capacitance; R: loss resistance of antenna.

According to FIG. 7 the resonance cavity of the transmitting antenna is implemented to have the possibility of adjusting its resonance frequency on any side of the resonance cavity of the receiving antenna. The adjustment may be made using pot trimming.

Accordingly, in some embodiments, the receiver antenna is designed with resonance (Q) factor to trap the displacement or pulling margin of the transmitter frequency which is caused by re-radiation process from the irradiated COVID-19 biological molecular structure delayed by relaxation time.

In some embodiments, a calibration procedure for the device manipulates the transmitting antenna resonance frequency by trimming a pot and displacing it in the proper operation region.

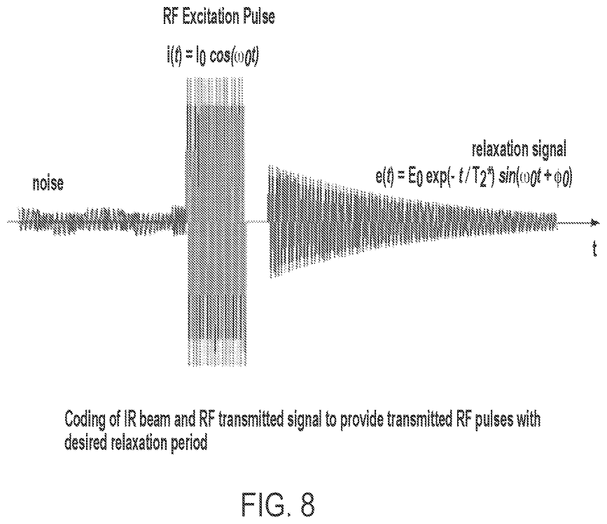

According to some embodiments, the excitation comprises combined Infrared (IR) and Radiofrequency (RF) electromagnetic fields. Both IR beam and RF transmitted signal are coded in a way to give the transmitted RF pulses the proper relaxation period as shown in FIG. 8. This coherency in the excitation method for both types of energy boosts the energy value induced in the COVID-19 molecular structures more efficiently and drives the structures to resonate and back relax to their initial energy state in a characteristic manner that can be detected with a device configured in accordance with some embodiments.

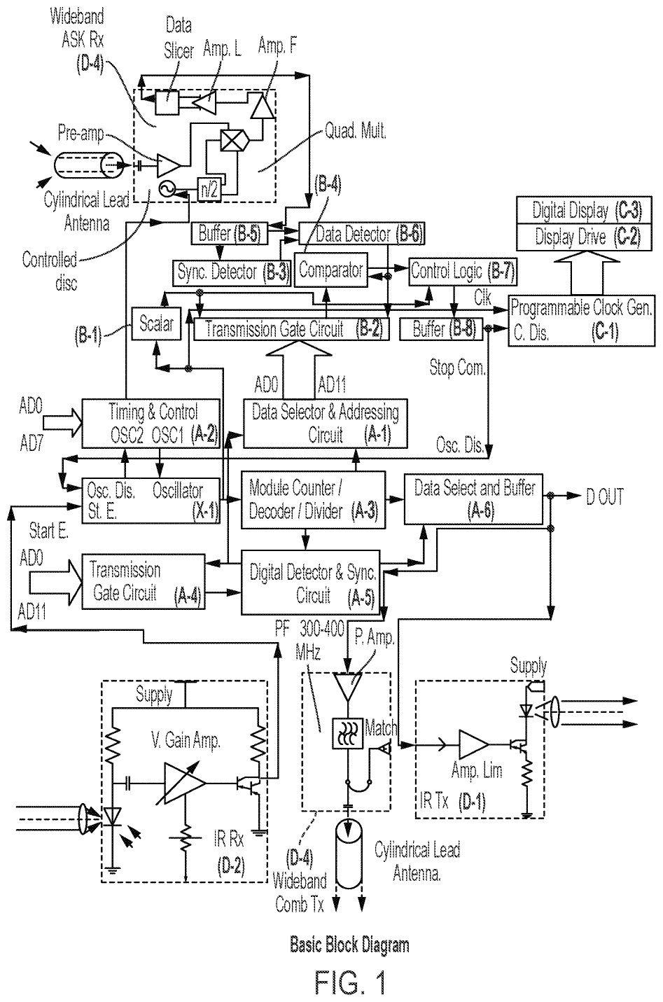

FIG. 1 illustrates a schematic block diagram of a device constructed in accordance with some embodiments.

According to some embodiments, the IR receiver shown in FIG. 1 (block D-2) serves to detect the first reflected ray to initiate the starting of the measurement process after correlating positive true IR beam detection with the positive true RF receiver signal detection.

According to some embodiments, the RF receiver, after positively true detection of the combined excitation signals, slices the data digitally, then operates the digital signal processing blocks (A's and B's of FIG. 1). These blocks perform signal extracting and decoding processes in accordance with some embodiments. In some embodiments, the digital signal processing blocks also perform encoding operations for both the IR and RFgenerators.

According to some embodiments, the coherent excitation of the COVID-19 molecular structure in sample under test will increase the energy content of the atomic and molecular particles and with time the resonance condition will be reached. The receiver may be configured to detect this temporary displacement in the transmitter carrier frequency to stop the measurement operation.

According to some embodiments, a special count circuit is designed (FIG. 1: block C-1) to correlate the measurement time period with correction table implemented within the processing blocks to determine the correct detection threshold.

According to some embodiments, the whole system blocks (A's, B's, C-1, and C-2) are implemented on a single Field Programmable Gate Array (FPGA) chip. The controlling software program may also be downloaded on the same chip.

A device designed in accordance with some embodiments was tested over the past year on more than 800 patients, each at different test conditions and the results were encouraging with an error in reading of less than 2%.

As described above, a non-invasive COVID-19 detection methodology using combined excitation techniques can be easily used and have size and weight suitable for use. Additionally, some embodiments can be used on a domestic level for periodic self-test non-invasively. In addition, additional cost is not required for PCR chemical diagnostic agents, or other cost.

The foregoing embodiment and advantages are merely exemplary and are not to be construed as limiting to embodiments of the invention.

The description of the embodiments of the present invention is intended to be illustrative, and not to limit the scope of the claims, and many alternatives, modifications, and variations will be apparent to those skilled in the art.

Accordingly, some embodiments have been made to solve some detection problems associated with the classical procedure used to detect infected people or substances with COVID-19 viruses. Some embodiments provide a non-invasive COVID-19 detection procedure using combined excitation techniques of both electromagnetic wave and IR energy which does not require any other agent. Users can precisely and accurately perform periodic self-test in their homes and with high level of certainty thereof.

Accordingly, some embodiments can be operated by untrained personnel in non-institutional locations. The foregoing statements of features are not exhaustive and serve merely to illustrate some of the many features of some embodiments of the invention.

Accordingly, some embodiments implement a non-invasive COVID-19 sensor using a secondary resonance cavity method; the transmitting and receiving microstrip resonance element are implemented to fulfill this feature, including a unit for measuring relaxation time of the biological molecular structure of COVID-19 virus via applying combined RF and IR waves.

Accordingly, a circuit unit generating the electromagnetic waves (FIG. 1 block D-3) that can be fine-tuned the measuring unit and hence determining the positive true detection threshold.

For this reason, the transmitting resonance cavity (micro-strip antenna backed by cylindrical resonator cavity) is designed with very high quality factor (Q) to maintain very narrow bandwidth and with directivity perpendicular to the horizontal plane of the invented device.

Accordingly, a circuit unit is implemented (FIG. 1, block D-4) in some embodiments to detect the electromagnetic waves in quadratic demodulation and slicing the demodulated data to be ready for processing.

For this reason, the receiving resonance cavity (micro-strip antenna backed by cylindrical resonator cavity) in some embodiments is designed with moderate quality factor (Q) to maintain wider bandwidth and with directivity perpendicular to the horizontal plane of the invented device.

Accordingly, some embodiments utilize digital processing circuitries (FIG. 1, blocks A's; B's; and C's) which are implemented to correlate the COVID-19 virus relaxation time stored signatures with the detected ones to reach precise decision. The drive controller and the calculating units may be embodied as computer-executable instructions inside a single chip Field Programmable Gate Array (FPGA).

Some embodiments implement an IR near visible source for radiating 810-850 nm IR beam onto the human body under investigation; a switching module (FIG. 1 block D-1) capable of generating IR beam in coherence with the baseband modulating encoder signal of the electromagnetic field generator. The IR unit includes sensor module for receiving the reflected first light ray from the body under investigation. The IR (source and receiver) are contained within the device.

Some embodiments are implemented as a handheld device that also includes a circuit unit with which a calibration procedure to fit resonance characteristics of both resonance cavities can be achieved.

The described non-invasive methodology for detecting the infected body with COVID-19 virus can secure the required measures and improve the precision and accuracy of such procedure.

SARS-COV-2 is classified within the genus Betacoronavirus (subgenus Sarbecovirus) in the family Coronaviridae (subfamily Orthocoronavirinae), a family of single-strand positive-sense RNA viruses [Sanjuán R, Domingo-Calap P. Mechanisms of viral mutation. Cell Mol Life Sci. 2016; 73:4433-48. doi: 10.1007/s00018-016-2299-6]. The International Committee on the Taxonomy of Viruses (ICTV) currently considers SARS-COV-2 as belonging to the species Severe acute respiratory syndrome-related coronavirus, along with SARS-COV and closely related viruses sampled from non-human species [Coronaviridae Study Group of the International Committee on Taxonomy of Viruses. The species severe acute respiratory syndrome-related coronavirus: classifying 2019-nCov and naming it SARS-COV-2. Nat Microbiol. 2020; 5:536-44. doi: 10.1038/s41564-020-0695-z]. See also, SARS-COV-2 NCBI Genome Reference Sequence (NC_045512), incorporated by reference. SARS-COV-2 sequences and information available at the National Center for Biotechnology Information (NCBI) (see: https://www.ncbi.nlm.nih.gov/). The reference strain of SARS-COV-2. Wuhan-Hu-1 (GenBank accession MN908947), was sampled from a patient in Wuhan, China, on 26 Dec. 2019 [Wu F, Zhao S, Yu B, Chen Y-M, Wang W, Song Z-G et al. A new coronavirus associated with human respiratory disease in China. Nature. 2020; 579:265-9. doi: 10.1038/s41586-020-2008-3]. That genome is 29 903 nucleotides (nt) in length and comprises a gene order of similar structure to that seen in other coronaviruses: 5′-replicase ORF1ab-S-E-M-N-3′. The predicted replicase ORF1ab gene of Wuhan-Hu-1 is 21 291 nt in length. The ORF1ab polyprotein is predicted to be cleaved into 16 nonstructural proteins. ORF1ab is followed by a number of downstream open reading frames (ORFs). These include the predicted S (spike), ORF3a, E (envelope), M (membrane) and N (nucleocapsid) genes of lengths 3822, 828, 228, 669 and 1260 nt, respectively, each a potential target (e.g., marker) for detection. Like SARS-COV, Wuhan-Hu-1 also contains a predicted ORF8 gene (366 nt in length) located between the M and N genes. Finally, the 5′ and 3′ terminal sequences of Wuhan-Hu-1 are also typical of betacoronaviruses and have lengths of 265 nt and 229 nt, respectively.

Virus genomic sequence data can be important in helping to identify virus proteins that are likely to be strongly antigenic, and to indicate how these antigens can be produced for serological assays. Peptide screening has indicated that the four SARS-COV-2 structural proteins, S, E, M and N, are likely to be the most strongly antigenic [Ren Y. Zhou Z, Liu J, Lin L, Li S. Wang H et al. A strategy for searching antigenic regions in the SARS-COV spike protein. Genomics Proteomics Bioinformatics. 2003; 1:207-15. doi: 10.1016/s1672-0229(03)01026-x; Kumar S. Maurya V K, Prasad A K, Bhatt M L B, Saxena S K. Structural, glycosylation and antigenic variation between 2019 novel coronavirus (2019-nCOV) and SARS coronavirus (SARS-COV). Virusdisease. 2020:1-9. doi: 10.1007/s13337-020-00571-5], and thus each of SARS-COV-2 structural proteins, S. E. M and N are potential detection markers. Thus, these processes and detection devices rely on understanding the genomic sequence and structure of SARS-COV-2 proteins, nucleic acids (e.g., DNA, RNA. mRNA, any genetic material), and variants thereof, as well as unique molecules relating to SARS-COV-2 that may be found in one or more of serum, blood, respiratory samples (e.g., nasooropharyngeal swabs, sputum, bronchial lavage fluid), saliva, oral fluids, fecal and anal swabs, feces, tissue samples, viral isolates from clinical samples (e.g., cell culture, animal model, and the like).

### EMBODIMENTS

Embodiments of the invention include the devices and methods described generally herein, and including but not limited to:
1. Device and methodology non-invasively for remotely detecting a marker of disease or disorder in a subject sample, the device comprises:
   IR transmitter with all associated chopping circuits (FIG. 1 block D-1).
   IR receiver with all filtering circuits (FIG. 1 block D-2).
   An RF energy source arranged to generate a range of VHF and UHF frequencies (FIG. 1 block D-3).
   Transmitting resonance cavity antenna coupled to RF energy source and implemented to transmit the generated RF energy into the body under test.
   Quadrature receiver circuit with all limiting and filtering circuits to detect the specially implemented transmitted codes (FIG. 1 block D-4).
   Receiving resonance cavity antenna arranged to receive the coupled part of the RF energy transmitted through under-test the body.
   A signal processor arranged to determine the resonant frequency of the received microwave energy; and
   A data processor arranged to issue an accurate decision on the status of the body under test according to the time of occurrence of the relaxed resonance frequency.
2. Device according to embodiment 1, wherein the IR transmitter is implemented to transmit its beam modulated according to the generated code sequence.
3. Device according to embodiments 1 or 2, wherein the IR transmitter is implemented to generate its beam at low power level (less than 1 mW) to maintain gradual excitation of the molecules and atoms of the under-test biological molecular structure.
4. Device according to any of embodiments 1-3, wherein the IR receiver is implemented to detect the reflected coded beam. The IR receiver comprises all required filters, limiters; and automatic gain control amplifier circuits.
5. Device according to any of embodiments 1-4, wherein the IR receiver is implemented to detect the first reflected coded beam to initiate the measuring process.
6. Device according to any of embodiments 1-5, wherein the RF energy source is arranged to generate RF energy over a range of frequencies within the resonant frequency of the under-test biological molecular structure.
7. Device according to any of embodiments 1-6, wherein the RF energy source is implemented as fine-digitally tuned oscillator to control the generated frequency precisely.
8. Device according to any of embodiments 1-6, wherein the RF energy source comprises a quadrature frequency multiplier to modulate the output RF energy according to the incoming code sequence.
9. Device according to embodiment 7, wherein the RF energy source comprises a quadrature frequency multiplier to modulate the output RF energy according to the incoming code sequence.
10. Device according to any of embodiments 1-9, wherein transmitting resonance cavity-antenna and the receiving resonance cavity-antenna comprise microstrip antennas enclosed in cylindrical resonator, each antenna has a radiating U-shaped resonating element and an RF feed line.
11. Device according to any of embodiments 1-10, wherein both transmitting resonance cavity antenna and the receiving resonance cavity antenna have radiation pattern perpendicular to the device aperture plane.
12. Device according to any of embodiments 1-11, wherein the separation between the resonance frequency of the transmitting antenna and the resonance frequency of the receiving antenna is designed to have the minimum value to keep the receiving one just blind to the transmitting one under no test condition.
13. Device according to any of embodiments 1-12, wherein the Q factor of the transmitting resonance cavity antenna is very high and the Q factor of the receiving resonance cavity antenna much lower than the first one.
14. Device according to any of embodiments 1-13, wherein the transmitting resonance frequency can be adjusted on any side of the response curve of the receiving one by simply trimming a pot to select minimum separation between both antennas to keep them blind to each other.
15. Device according to any of embodiments 1-14, wherein for both transmitting and receiving antennas, the RF feed line comprises a microstrip connected to the radiating or receiving element.
16. Device according to any of embodiments 1-15, wherein quadrature receiver circuit with all limiting and filtering circuits to detect the specially implemented transmitted codes also comprises a data slicer circuit to produce the digital sequence which is required for further processing.
17. Device according to any of embodiments 1-16, wherein the signal processor (FIG. 1 blocks A's, B's, C-1, and C-2) is arranged to generate the coding sequence via pre-selected address AD₀-AD₁₁. This sequence is essential for the operation of the IR transmitter, RF generator and the received-signal correlation process.
18. Device according to any of embodiments 1-17, wherein the signal processor (FIG. 1 blocks A's, B's, C-1, and C-2) is arranged to detect the first reflected IR ray to start the programmable counter.
19. Device according to any of embodiments 1-18, wherein the programmable counter is used to translate the count between start of excitation-to-occurrence of resonance of the under-test biological molecular structure to an address to the built-in lockup table that has been arranged in accordance to second order Cole-Cole algorithm.
20. Device according to any of embodiments 1-19, wherein the digital parts (FIG. 1 blocks A's, B's, C-1, and C-2) are implemented on a single Field Programmable Array (FPGA) chip. The controlling software and the calibrating lookup table are also downloaded on the same chip.
21. Device according to any of embodiments 1-20, wherein the parts of FIG. 1 (blocks A's, B's, C's, C's, and X-1) are implemented inside small portable plastic case.
22. Device according to embodiment any of embodiments 1-21, wherein the IR transmitter is arranged in a way such that the IR diode beam is emerging through a hole in the device aperture plane.
23. Device according to any of embodiments 1-22, wherein the IR detector is arranged in a way such that it only sees the beam reflecting from the under-test biological molecular structure through a different hole from and adjacent to that of the IR transmitter.
24. Device according to any of embodiments 1-23, wherein the test methodology is to non-invasively placing the under-test biological molecular structure perpendicular to the device aperture plane at a maximum distance of 2-meters. This methodology produces the optimum interaction to sense the pulling effect by the biological tissue under test.
25. A marker detection device, comprising:
at least one transmitter configured to transmit infrared (IR) electromagnetic radiation and radiofrequency (RF) energy into a sample;
receiving circuitry configured to detect RF energy emitted from the sample in response to stimulation of the sample with the transmitted IR electromagnetic radiation and RF energy;
signal processing circuitry configured to analyze the RF energy detected by the receiving circuitry to determine a resonant frequency; and
at least one computer processor configured to identify the presence of a virus in the sample based, at least in part, on the determined resonant frequency.
26. The embodiment of any of embodiments 1-25, wherein the marker is a small molecule compound, metabolite, protein, gene, ribonucleic acid (RNA), mRNA, bacteria, or virus, including wild-type and mutant forms.
27. The embodiment of any of embodiments 1-26, wherein the marker is associated with a disease or disorder.
28. A method for detecting non-invasively for remotely detecting a marker of disease or disorder, the method comprising testing an object using a device of any of embodiments 1-27.
29. The method of embodiment 28, wherein the object is a person, sample obtained from a person, animal, sample obtained from an animal, or inanimate object or surface.
30. The method of embodiment 28 or 29, wherein the testing comprises subjecting the object to transmission of an RF energy source.
31. The method of any one of embodiments 28-30, wherein the testing comprises subjecting the object to transmission of an RF energy source and receiving an RF energy signal from the object.
32. The method of any one of embodiments 28-30, wherein the analysis of subjecting the object to transmission of an RF energy source and receiving an RF energy signal from the object indicates the presence of the marker of disease or disorder.
33. The method of any one of embodiments 28-30, wherein the analysis of subjecting the object to transmission of an RF energy source and receiving an RF energy signal from the object indicates the absence of the marker of disease or disorder.
34. Use of electromagnetic waves for detection of SARS-COV-2 or any of its components.
35. Use of combination of Infra-red and Radio Frequency for detection of SARS-Cov-2 or any of its components.
36. Use of electromagnetic waves (combination of Infra-red and Radio Frequency) for detection of any infectious agent, including viruses, bacteria, yeast, amoeba, fungi, parasites, prions, etc.
37. Use of electromagnetic waves (combination of Infra-red and Radio Frequency) for detection of any procaryote or eucaryote cell type (such as human cells, animal cells, plant cells, cancer cells, specific tissue cells, bacteria, viruses, fungi, parasites, yeast).
38. Non-invasive, non-touch, detection of SARS-COV-2 or any of its components, anywhere.
39. Immediate detection of SARS-COV-2 or any of its components (e.g., less than 5 minutes, less, then 3 minutes, less than 1 minute, less than 30 seconds, less than 20 seconds, less than 10 seconds, less than 5 seconds).
40. Immediate detection of SARS-COV-2 or any of its components from 0-4 meters away (e.g., less than 4 meters, less than 3 meters, less than 2 meters, less than 1 meter, less than 0.5 meters, less than 0.25 meters, less than 0.1 meters).
41. Detection of SARS-COV-2 or any of its components with >99% specificity [performance].
42. Detection of SARS-COV-2 or any of its components with >99% sensitivity [performance].
43. Detection of SARS-COV-2 specific components in a specific and sensitive manner, including proteins (such as E, N, M, and S proteins) and mRNA (genetic material specific to this virus only).
44. Immediate detection of SARS-COV-2 or any of its components in any human sample, including nasal swabs, oral swabs, sputum, saliva, bronchoalveolar lavage (lung aspirate), and any human samples, whether liquid or solid tissue from biopsies or autopsies.
45. Immediate detection of people infected with SARS-COV-2 (carriers) in a non-invasive manner.
46. Immediate detection of people who are infected with SARS-COV-2 AND who are infectious (transmitters of infection) in a non-invasive manner.
47. Immediate detection of SARS-COV-2 in air coughed or sneezed or blown out of mouth or nose of infected people (to see who is shedding off the virus).
48. Immediate detection of SARS-COV-2 on the outside of human body (such as on hands, face, clothes, shoe bottom, etc.).
49. Immediate detection of SARS-COV-2 on contaminated objects (e.g. inanimate objects or surfaces, including for example, door handles, mobile phones, car door handles and steering wheels, calculators, chairs, tables, computer mouse, keyboards, appliances, buttons, knobs, handles, seats, lavatories, kitchens, tables, etc.).
50. Immediate detection of very low copy numbers of SARS-COV-2 inside and outside the human body.
51. Use of the device to verify RT-PCR results of COVID19 infection.
52. Use of the device to locate SARS-COV-2 in specific organs in the body of humans and animals.
53. Detection of SARS-COV-2 on contaminated foods (important for food suppliers, import, export).
54. Use of the device as a screening device for humans, animals, and objects, prior to entering specific areas (airports, train stations, stadiums, hospitals, schools, universities, restaurants, cruise line ships, malls, etc.) to create clean or safe zones (e.g., “bubbles”), that is areas relatively free from, or having reduced levels of, disease or disease pathogens.
55. A method for detecting non-invasively for remotely detecting the COVID-19 virus and variants thereof (e.g., SARS-COV-2; variants B.1.1.7 (UK), 1.315 (South Africa). B.1.1.529, and P.1 Brazil)), the method comprising testing an object using a device of any of embodiments 1-24.
56. The method of embodiment 55, wherein the object is a person, sample obtained from a person, animal, sample obtained from an animal, or inanimate object or surface.
57. The method of embodiment 55 or 56, wherein the testing comprises subjecting the object to transmission of an RF energy source.
58. The method of any one of embodiments 55-57, wherein the testing comprises subjecting the object to transmission of an RF energy source and receiving an RF energy signal from the object.
59. The method of any one of embodiments 55-58, wherein the analysis of subjecting the object to transmission of an RF energy source and receiving an RF energy signal from the object indicates the presence of the COVID-19 virus and variants thereof (e.g., SARS-COV-2; variants B.1.1.7 (UK). 1.315 (South Africa). B.1.1.529, and P.1 Brazil)).
60. The method of any one of embodiment 55-58, wherein the analysis of subjecting the object to transmission of an RF energy source and receiving an RF energy signal from the object indicates the absence of the COVID-19 virus and variants thereof (e.g., SARS-COV-2; variants B.1.1.7 (UK), 1.315 (South Africa). B.1.1.529, and P.1 Brazil)).

The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof. All references cited herein are incorporated by reference in their entirety.

What is claimed is:

1. Device and methodology non-invasively for remotely detecting the COVID-19 virus and variants thereof (e.g., SARS-COV-2; variants B.1.1.7 (UK), 1.315 (South Africa), and P.1 Brazil)), the device comprises:
IR transmitter with all associated chopping circuits (FIG. 1 block D-1).
IR receiver with all filtering circuits (FIG. 1 block D-2).
An RF energy source arranged to generate a range of VHF and UHF frequencies (FIG. 1 block D-3).
Transmitting resonance cavity antenna coupled to RF energy source and implemented to transmit the generated RF energy into the body under test.
Quadrature receiver circuit with all limiting and filtering circuits to detect the specially implemented transmitted codes (FIG. 1 block D-4).
Receiving resonance cavity antenna arranged to receive the coupled part of the RF energy transmitted through under-test the body.
A signal processor arranged to determine the resonant frequency of the received microwave energy; and
A data processor arranged to issue an accurate decision on the status of the body under test according to the time of occurrence of the relaxed resonance frequency.

2. Device according to claim 1, wherein the IR transmitter is implemented to transmit its beam modulated according to the generated code sequence.

3. Device according to claim 2, wherein the IR transmitter is implemented to generate its beam at low power level (less than 1 mW) to maintain gradual excitation of the molecules and atoms of the under-test biological molecular structure.

4. Device according to claim 2, wherein the IR receiver is implemented to detect the reflected coded beam. The IR receiver comprises all required filters, limiters; and automatic gain control amplifier circuits.

5. Device according to claim 4, wherein the IR receiver is implemented to detect the first reflected coded beam to initiate the measuring process.

6. Device according to claim 1, wherein the RF energy source is arranged to generate RF energy over a range of frequencies within the resonant frequency of the under-test biological molecular structure.

7. Device according to claim 6, wherein the RF energy source is implemented as fine-digitally tuned oscillator to control the generated frequency precisely.

8. Device according to claim 7; wherein the RF energy source comprises a quadrature frequency multiplier to modulate the output RF energy according to the incoming code sequence.

9. Device according to claim 1, wherein transmitting resonance cavity-antenna and the receiving resonance cavity-antenna comprise microstrip antennas enclosed in cylindrical resonator, each antenna has a radiating U-shaped resonating element and an RF feed line.

10. Device according to claim 9, wherein both transmitting resonance cavity antenna and the receiving resonance cavity antenna have radiation pattern perpendicular to the device aperture plane.

11. Device according to claim 10, wherein the separation between the resonance frequency of the transmitting antenna and the resonance frequency of the receiving antenna is designed to have the minimum value to keep the receiving one just blind to the transmitting one under no test condition.

12. Device according to claim 10, wherein the Q factor of the transmitting resonance cavity antenna is very high and the Q factor of the receiving resonance cavity antenna much lower than the first one.

13. Device according to claim 9, wherein the transmitting resonance frequency can be adjusted on any side of the response curve of the receiving one by simply trimming a pot to select minimum separation between both antennas to keep them blind to each other.

14. Device according to claim 9, wherein for both transmitting and receiving antennas, the RF feed line comprises a microstrip connected to the radiating or receiving element.

15. Device according to claim 1, wherein quadrature receiver circuit with all limiting and filtering circuits to detect the specially implemented transmitted codes also comprises a data slicer circuit to produce the digital sequence which is required for further processing.

16. Device according to claim 1, wherein the signal processor (FIG. 1 blocks A's, B's, C-1, and C-2) is arranged to generate the coding sequence via pre-selected address AD₀-AD₁₁. This sequence is essential for the operation of the IR transmitter, RF generator and the received-signal correlation process.

17. Device according to claim 16, wherein the signal processor (FIG. 1 blocks A's, B's, C-1, and C-2) is arranged to detect the first reflected IR ray to start the programmable counter.

18. Device according to claim 17, Wherein the programmable counter is used to translate the count between start of excitation-to-occurrence of resonance of the under-test biological molecular structure to an address to the built-in lockup table that has been arranged in accordance to second order Cole-Cole algorithm.

19. Device according to claim 1, wherein the digital parts (FIG. 1 blocks A's, B's, C-1, and C-2) are implemented on a single Field Programmable Array (FPGA) chip. The controlling software and the calibrating lookup table are also downloaded on the same chip.

20. Device according to claim 1, wherein the parts of FIG. 1 (blocks A's, B's, C's, C's, and X-1) are implemented inside small portable plastic case.

21. Device according to claim 2, wherein the IR transmitter is arranged in a way such that the IR diode beam is emerging through a hole in the device aperture plane.

22. Device according to claim 4, wherein the IR detector is arranged in a way such that it only sees the beam reflecting from the under-test biological molecular structure through a different hole from and adjacent to that of the IR transmitter.

23. Device according to claim 1, wherein the test methodology is to non-invasively placing the under-test biological molecular structure perpendicular to the device aperture plane at a maximum distance of 2-meters. This methodology produces the optimum interaction to sense the pulling effect by the biological tissue under test.

24. A virus detection device, comprising:
at least one transmitter configured to transmit infrared (IR) electromagnetic radiation and radiofrequency (RF) energy into a sample;
receiving circuitry configured to detect RF energy emitted from the sample in response to stimulation of the sample with the transmitted IR electromagnetic radiation and RF energy;
signal processing circuitry configured to analyze the RF energy detected by the receiving circuitry to determine a resonant frequency; and
at least one computer processor configured to identify the presence of a virus in the sample based, at least in part, on the determined resonant frequency.

25. A method for detecting non-invasively for remotely detecting the COVID-19 virus and variants thereof (e.g., SARS-COV-2; variants B.1.1.7 (UK), 1.315 (South Africa), B.1.1.529, and P.1 Brazil)), the method comprising testing an object using a device of any of claims 1-24.

26. The method of claim 25, wherein the object is a person, sample obtained from a person, animal, sample obtained from an animal, or inanimate object or surface.

27. The method of claim 25 or 26, wherein the testing comprises subjecting the object to transmission of an RF energy source.

28. The method of any one of claims 25-27, wherein the testing comprises subjecting the object to transmission of an RF energy source and receiving an RF energy signal from the object.

29. The method of any one of claims 25-28, wherein the analysis of subjecting the object to transmission of an RF energy source and receiving an RF energy signal from the object indicates the presence of the COVID-19 virus and variants thereof (e.g., SARS-COV-2; variants B.1.1.7 (UK), 1.315 (South Africa), and P.1 Brazil)).

30. The method of any one of claims 25-28, wherein the analysis of subjecting the object to transmission of an RF energy source and receiving an RF energy signal from the object indicates the absence of the COVID-19 virus and variants thereof (e.g., SARS-COV-2; variants B.1.1.7 (UK), 1.315 (South Africa), and P.1 Brazil)).

31. A method for detecting non-invasively for remotely detecting the COVID-19 virus and variants thereof (e.g., SARS-COV-2; variants B. 1.1.7 (UK), 1.315 (South Africa), and P.1 Brazil)), the method comprising testing an object using a device of any of claims 1-24.
